(19) United States
(12) Patent Application Publication
(10) Pub. No.: US 2022/0296685 A1
(43) Pub. Date: Sep. 22, 2022

(54) Uricase Derivative Co-conjugated With Fatty acid-linked PEG and optionally, Alkoxy PEG

(71) Applicant: SunBio, Inc., Gyeonggi-do (KR)

(72) Inventors: Kwang Nho, Gyeonggi-do (KR); Minjung Ahn, Gyeonggi-do (KR); Byunghee Sohn, Seoul (KR); Changmin Hyun, Gyeonggi-do (KR); Junghoon Lee, Gyeonggi-do (KR)

(21) Appl. No.: 17/696,374

(22) Filed: Mar. 16, 2022

Related U.S. Application Data
(60) Provisional application No. 63/162,342, filed on Mar. 17, 2021.

(51) Int. Cl. A61K 38/44; A61K 47/54; A61K 47/60; C12N 9/06

(52) U.S. Cl. CPC A61K 38/44; A61K 47/54; A61K 47/60; C12N 9/0048; C12Y 107/03003

(57) ABSTRACT

A uricase derivative co-conjugated with fatty acid-linked polyethylene glycol derivatives and optionally, alkoxy polyethylene glycol derivatives is presented. The uricase may be intramolecularly crosslinked. The uricase derivative of the present invention is intended for use as a treatment for gout or hyperuricemia.

### FIELD OF THE INVENTION

The present invention relates to novel formulation of PEG-uricase. In particular, the uricase is co-conjugated with multiple strands of fatty acid-linked polyethylene glycols and optionally, alkoxy polyethylene glycols.

### Definitions

The definition of terms is provided below to facilitate the understanding of the invention.

The term “polyethylene glycol” (PEG) refers to polymers of the general chemical formula HO(CH₂CH₂O)ₙH, where n is an integer greater than 1. The group represented as —(CH₂CH₂O)ₙ— is called the PEG backbone. The end group can be H or alkoxy of the general formula CH₃(CH₂)ₙO— where n is 0 or any integer. The most common structure of PEG is linear, such as revealed in methoxy PEG or hydroxy PEG. However, the PEG can be of a diverse structure, including that of multi-armed PEG, branched PEG, or PEG with degradable linkages built within the backbone.

As used herein, “fatty acid” is aliphatic monocarboxylic acid derived from or contained in esterified form in an animal or vegetable fat, oil, or wax. Saturated fatty acid refers to a type of fatty acid in which all carbon-carbon bonds are single bonds, and unsaturated fatty acid refers to a type of fatty acid having double or triple bonds.

The term “alkoxy-PEG” refers to alkoxy PEG, having a structure of RO(CH₂CH₂O)ₙH, where R is alkyl, including, in a non-limiting sense, methoxy-PEG, ethoxy-PEG, propoxy-PEG, etc.

The term “FA-PEG” or “fatty acid-PEG” refers to fatty acid-linked PEG where one molecule of fatty acid is conjugated to one molecule of PEG. Specifically, it refers to fatty acid-linked PEG prepared by reacting a fatty acid derivative which is prepared from fatty acid with PEG derivatives, and more specifically, in which PEG and carbonyl of fatty acid are linked via specific linkages (e.g., linkages comprising amine or sulfur).

The term “PEGylate” or “PEGylation” refers to the art of conjugating PEG to a target protein, such as uricase.

The term “FA-PEGylate” or “FA-PEGylation” refers to the art of conjugating FA-PEG to a target protein, such as uricase.

The term “alkoxy-PEG derivative” or “functional alkoxy-PEG” refers to PEG having a particular functional group at the end of the PEG, with the functional group capable of reacting readily with an electrophile or a nucleophile on the target molecule, such as uricase.

The term “FA-PEG derivative” or “functional FA-PEG” refers to FA-PEG having a particular functional group at the end of the FA-PEG, with the functional group capable of reacting readily with an electrophile or a nucleophile on the target molecule, such as uricase.

The term “PEG-uricase” refers to a uricase that is PEGylated.

The term “FA-PEG-uricase” refers to a FA-PEG-conjugated uricase, wherein a molecule of uricase is conjugated with one or more molecules of FA-PEG.

The term “[FA-PEG]ₚ-Uricase” refers to a uricase conjugated with FA-PEG wherein p denotes the number of FA-PEGs conjugated to the uricase.

The term “[FA-PEG]ₚ-Uricase-[alkoxy-PEG]_{q}” refers to a uricase co-conjugated with FA-PEG and alkoxy PEG wherein p denotes the number of FA-PEG conjugated, and q denotes the number of alkoxy PEGs conjugated to the uricase.

The term “[Stearic-PEG]ₚ-Uricase” refers to a uricase conjugated with stearic-PEG wherein p denotes the number of stearic-PEGs conjugated to the uricase.

The term “[Stearic-PEG]ₚ-Uricase-[alkoxy-PEG]_{q}” refers to a uricase co-conjugated with stearic-PEG and alkoxy PEG wherein p denotes the number of stearic-PEGs conjugated, and q denotes the number of alkoxy PEG conjugated to the uricase.

The term “xUricase” refers to a uricase that is intramolecularly crosslinked.

The term “FA-PEG-xUricase” refers to a FA-PEG-conjugated xUricase, wherein a molecule of uricase is conjugated with one or more molecules of FA-PEGs.

The term “[FA-PEG]ₚ-xUricase” refers to a xUricase conjugated with FA-PEGs wherein p denotes the number of FA-PEG conjugated to the uricase.

The term “[FA-PEG]ₚ-xUricase-[alkoxy-PEG]_{q}” refers to a xUricase co-conjugated with FA-PEG and alkoxy PEG wherein p denotes the number of FA-PEG conjugated, and q denotes the number of alkoxy PEG conjugated to the uricase.

The term “[Stearic-PEG]ₚ-xUricase” refers to a xUricase conjugated with stearic-PEG wherein p denotes the number of stearic-PEG conjugated to the uricase.

The term “[Stearic-PEG]ₚ-xUricase-[alkoxy-PEG]_{q}” refers to a xUricase co-conjugated with stearic-PEG and alkoxy-PEG wherein p denotes the number of stearic-PEG conjugated, and q denotes the number of alkoxy PEG conjugated to the uricase.

The term “conjugation”, “conjugating” or “be conjugated” refers to forming a covalent or direct linkage with desired parts (e.g. in the present invention, it means FA-PEG or alkoxy-PEG) in the resulting uricase derivative by reaction with uricase.

The term “alkyl” refers to a linear or branched, saturated hydrocarbon chain radical, including for example and in a non-limiting sense, ethyl, n-butyl, t-butyl, n-pentyl, octyl, dodecyl, octadecyl, etc.

The term “alkenyl” refers to a linear or branched, unsaturated hydrocarbon chain radical which includes one or more unsaturated bonds (e.g. double or triple bond), including for example and in a non-limiting sense, propenyl, butenyl, octenyl, dodecenyl, octadecenyl, etc.

The term “alkylene” refers to a divalent alkyl group and the term “alkenylene” refers to a divalent alkenyl group.

The term “alkoxy” refers to an —O—R group, wherein R is alkyl, alkenyl, or substituted alkyl or alkenyl, etc (e.g., methoxy or ethoxy).

The term “amide” refers to a divalent group which has the formula —N(R)CO—, wherein R is H, alkyl, alkenyl, aryl, arylalkyl, or the like.

The term “carbamate” and “urethane” refers to a divalent group which has the formula —OC(O)NR—, wherein R is H, alkyl, alkenyl, aryl, arylalkyl, or the like.

The term “carbonate” refers to a divalent group which has the formula —OC(O)O—.

The term “ester” refers to a divalent group which has the formula —OC(O)—.

The term “ether” refers a divalent group which has the formula —O—.

The term “carbonyl” refers to a divalent group which has the formula —C(O)—.

The term “ameliorating” or “ameliorate” refers to any indicia of success in the treatment of a pathology or condition, including any objective or subjective parameter, such as abatement, remission or diminishing of symptoms or an improvement in a patient's physical or mental well-being. Amelioration of symptoms can be based on objective or subjective parameters, including the results of a physical examination and/or a psychiatric evaluation.

The term “Pharmaceutical composition” refers to a composition comprising a compound of the invention and at least one component selected from the group comprising pharmaceutically acceptable adjuvants, carriers, diluents, excipients, fillers, preserving agents or suspending agents, depending on the nature of the mode of administration and dosage forms.

As used herein, “pharmaceutically acceptable carrier” includes any material, which when combined with the conjugate retains the conjugate's activity and is non-reactive with the subject's immune systems. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well-known conventional methods. Exemplary carriers are hypertonic sodium chloride and isotonic sodium chloride (e.g., phosphate buffered saline).

The term “treatment” or “treating” is to be understood as embracing prophylaxis and treatment or amelioration of symptoms of a disease and/or treatment of the cause of the disease. The term “prevention” or “preventing” is to be understood as all actions that inhibit or delay the development of a disease or disorder.

As used herein, terms such as “subject,” “patient,” and “mammal” are used interchangeably, and are exemplified by a human.

The meaning of other terminology used herein should be easily understood by any person skilled in the art.

### BACKGROUND OF THE INVENTION

Despite many effective treatments for gout or hyperuricemia, its management remains a challenge. Options for optimizing gout management may differ in different practice settings. Gout incidence is rising and it continues to be associated with increased mortality. Special consideration needs to be given to such populations as the elderly and those with renal and cardiovascular disease in gout management (Fields, T. R. et al.).

Treatment of acute attacks are usually treated with nonsteroid anti-inflammatory agents such as indomethacin, naproxen, sulindac or celecoxib. However, the major approach to long term prevention of gout and the complications of uric acid nephropathy is the use of uricosuric acids such as probenecid and/or inhibitors of xanthine oxidase, such as the xanthine derivative allopurinol and the newer nonnucleoside xanthine oxidase inhibitors such as febuxostat. Newer approaches to gout include use of lesinurad, a drug that inhibits the reabsorption of uric acid in the distal tubules of the kidney, and use of recombinant enzymes that metabolize uric acid such as pegloticase (Krystexxa), which is used in combination with xanthine oxidase inhibitors to treat severe gout, and rasburicase which is used to treat the hyperuricemia associated with tumor lysis syndrome induced by cancer chemotherapy. (Gout Medications, in LivefTox)

Uricases (urate oxidases) are enzymes that catalyze the oxidation of poorly soluble uric acid to soluble allantoin, which is more readily removed from body through renal excretion. Humans are genetically incapable of generating uricase. A high concentration of uric acid in the body may lead to hyperuricemia or gout. Uricase is a foreign protein in humans, administration of the unmodified uricase from native sources such as fungus or mammals other than humans is known to cause immunogenic responses and anaphylactic reactions in humans.

Conjugation of uricase with polyethylene glycol (PEG) has been used to increase intravascular uricase half-life and reduce immunogenicity. The attachment of PEG enables the PEG-uricase conjugate a protection of physical barrier against leucocytes as well as increased intravascular retention due to substantially increased molecular weight.

The following cited prior patents are all related to PEG-uricase.

U.S. Pat. No. 10,731,139 titled “Variant forms of urate oxidase and use thereof” authored by Hartman J. et al. discloses genetically modified proteins with uricolytic activity. Proteins comprising truncated urate oxidases and methods for producing them, including PEGylated proteins comprising truncated urate oxidase are described. In this patent, the uricase was conjugated with mPEG.

U.S. Pat. No. 10,160,958 titled “Variant forms of urate oxidase and use thereof” authored by Hartman J. et al. disclosed genetically modified proteins with uricolytic activity. Proteins comprising truncated urate oxidases and methods for producing them, including PEGylated proteins comprising truncated urate oxidase are described. In this patent, the uricase was conjugated with mPEG.

U.S. Pat. No. 9,926,538 titled “Variant forms of urate oxidase and use thereof” authored by Hartman J. et al. disclosed genetically modified proteins with uricolytic activity. Proteins comprising truncated urate oxidases and methods for producing them, including PEGylated proteins comprising truncated urate oxidase are described. In this patent, the uricase was conjugated with mPEG.

U.S. Pat. No. 9,926,537 titled “Variant forms of urate oxidase and use thereof” authored by Hartman J. et al. disclosed genetically modified proteins with uricolytic activity. Proteins comprising truncated urate oxidases and methods for producing them, including PEGylated proteins comprising truncated urate oxidase are described. In this patent, the uricase was conjugated with mPEG.

U.S. Pat. No. 9,885,024 titled “PEG-urate oxidase conjugates and use thereof” authored by Williams L. D. et al. discloses a naturally occurring or recombinant urate oxidase (uricase) covalently coupled to PEG, wherein an average of 2 to 10 strands of PEG are conjugated to each uricase subunit and the PEG has an average molecular weight between about 5 kDa and 100 kDa. In this patent, the uricase was conjugated with mPEG.

U.S. Pat. No. 9,670,467 titled “Variant forms of urate oxidase and use thereof” authored by Hartman J. et al. disclosed genetically modified proteins with uricolytic activity. Proteins comprising truncated urate oxidases and methods for producing them, including PEGylated proteins comprising truncated urate oxidase are described. In this patent, the uricase was conjugated with mPEG.

U.S. Pat. No. 9,193,967 titled “Pegylated analogue protein or canine urate oxidase, preparation method and use thereof” authored by Zhang C. et al. discloses a PEGylated analogue protein of canine urate oxidase, preparation method and use thereof. The analogue protein of canine urate oxidase is a canine urate oxidase, or a chimeric protein comprising part of the amino acid sequence of a canine urate oxidase and part of the amino acid sequence of a human urate oxidase, or a mutant protein thereof. In this patent, the uricase was conjugated with mPEG.

U.S. Pat. No. 9,017,980 titled “Variant forms of urate oxidase and use thereof” authored by Hartman J. et al. discloses genetically modified proteins with uricolytic activity. Proteins comprising truncated urate oxidases and methods for producing them, including PEGylated proteins comprising truncated urate oxidase are described. In this patent, the uricase was conjugated with mPEG.

U.S. Pat. No. 8,618,267 titled “PEG-urate oxidase conjugates and use thereof” authored by Williams L. D. et al. discloses a naturally occurring or recombinant urate oxidase (uricase) covalently coupled to PEG, wherein an average of 2 to 10 strands of PEG are conjugated to each uricase subunit and the PEG has an average molecular weight between about 5 kDa and 100 kDa. In this patent, the uricase was conjugated with mPEG.

U.S. Pat. No. 8,541,205 titled “Variant forms of urate oxidase and use thereof” authored by Hartman J. et al. discloses genetically modified proteins with uricolytic activity. Proteins comprising truncated urate oxidases and methods for producing them, including PEGylated proteins comprising truncated urate oxidase are described. In this patent, the uricase was conjugated with mPEG.

U.S. Pat. No. 8,188,224 titled “Variant forms of urate oxidase and use thereof” authored by Hartman J. et al. discloses genetically modified proteins with uricolytic activity. Proteins comprising truncated urate oxidases and methods for producing them, including PEGylated proteins comprising truncated urate oxidase are described. In this patent, the uricase was conjugated with mPEG.

U.S. Pat. No. 8,148,123 titled “Methods for lowering elevated uric acid levels using intravenous injections of PEG-uricase” authored by Hartman J. et al. discloses a method for lowering elevated uric acid levels in patients, and of administering to the patients an intravenous injection of PEG-uricase having a dosage from about 4 to about 12 mg. In this patent, the uricase was conjugated with mPEG.

U.S. Pat. No. 8,067,553 titled “PEG-urate oxidase conjugates and use thereof” authored by Williams L. D. et al. discloses a naturally occurring or recombinant urate oxidase (uricase) covalently coupled to PEG, wherein an average of 2 to 10 strands of PEG are conjugated to each uricase subunit and the PEG has an average molecular weight between about 5 kDa and 100 kDa. In this patent, the uricase was conjugated with mPEG.

U.S. Pat. No. 7,927,852 titled “Aggregate-free urate oxidase for preparation of non-immunogenic polymer conjugates” authored by Sherman M. R. et al. discloses a naturally occurring or recombinant protein, especially a mutein of porcine urate oxidase (uricase), that is essentially free of large aggregates can be rendered substantially non-immunogenic by conjugation with a sufficiently small number of strands of polymer such that the bioactivity of the protein is essentially retained in the conjugate. In this patent, the uricase was conjugated with mPEG.

U.S. Pat. No. 7,927,589 titled “PEG-urate oxidase conjugates and use thereof” authored by Williams L. D. et al. discloses a naturally occurring or recombinant urate oxidase (uricase) covalently coupled to PEG, wherein an average of 2 to 10 strands of PEG are conjugated to each uricase subunit and the PEG has an average molecular weight between about 5 kDa and 100 kDa. In this patent, the uricase was conjugated with mPEG.

U.S. Pat. No. 7,723,089 titled “PEG-urate oxidase conjugates and use thereof” authored by Williams L. D. et al. discloses a naturally occurring or recombinant urate oxidase (uricase) covalently coupled to PEG, wherein an average of 2 to 10 strands of PEG are conjugated to each uricase subunit and the PEG has an average molecular weight between about 5 kDa and 100 kDa. In this patent, the uricase was conjugated with mPEG.

U.S. Pat. No. 6,783,965 titled “Aggregate-free urate oxidase for preparation of non-immunogenic polymer conjugates” authored by Sherman M. R. et al. discloses a naturally occurring or recombinant protein, especially a mutein of porcine uricase, that is essentially free of large aggregates can be rendered substantially non-immunogenic by conjugation with a sufficiently small number of strands of polymer such that the bioactivity of the protein is essentially retained in the conjugate. In this patent, the uricase was conjugated with mPEG.

U.S. Pat. No. 6,576,235 titled “PEG-urate oxidase conjugates and use thereof” authored by Williams L. D. et al. discloses a naturally occurring or recombinant urate oxidase (uricase) covalently coupled to PEG, wherein an average of 2 to 10 strands of PEG are conjugated to each uricase subunit and the PEG has an average molecular weight between about 5 kDa and 100 kDa. In this patent, the uricase was conjugated with mPEG.

Reading all the above prior patents wherein the method of preparation of uricase derivative, specifically, PEG-uricase was disclosed, in all cases, methoxy PEG (mPEG) was used to pegylate uricase.

It is an object of the invention to provide novel uricase derivative, wherein the uricase is co-conjugated with multiple strands of fatty acid-linked PEG and optionally, alkoxy-PEG, which serve as a treatment for gout or hyperuricemia and have better therapeutic activity, and methods for the preparation of said uricase derivative.

### SUMMARY OF THE INVENTION

The present invention is a uricase derivative wherein the uricase is conjugated with fatty acid-linked PEG (FA-PEG) and optionally, alkoxy-PEG.

Fatty acids readily bind with intravascularly abundant serum albumin, thus the fatty acid-conjugated drugs, when bound to albumin, are known to facilitate protection from proteolytic degradation and renal clearance due to steric effect, leading to extended intravascular retention. PEG, when conjugated to drugs, has been known to elicit pharmacokinetic changes such as diminished immunogenic reactions, increased intravascular retention time and increased water solubility. By combining the characteristics of fatty acid and PEG on uricase, the present invention offers greater pharmacokinetic benefits, especially lowering the level of uric acid. The present invention provides a uricase derivative in which uricase or crosslinked uricase is conjugated with fatty acid-PEG (FA-PEG) derivatives and optionally, alkoxy-PEG derivatives.

The present invention also provides a pharmaceutical composition comprising the uricase derivative and a pharmaceutically acceptable carrier.

The present invention also provides a method of treating, preventing or alleviating a disease of gout or hyperuricemia, comprising administering the uricase derivative to a subject in need thereof.

Other aspects of the invention are described throughout the specification.

### BRIEF DESCRIPTION OF THE DRAWINGS

FIG. 1 shows in vivo uricase activity of [mPEG]₂₀-Uricase-[stearic-PEG]₂₀ (group A), and Native-Uricase (group B) according to Example 6.

### DETAILED DESCRIPTION OF THE INVENTION

The present invention relates to a uricase derivative in which uricase or crosslinked uricase is conjugated with fatty acid-PEG (FA-PEG) derivatives.

The present invention further relates to a uricase derivative in which uricase or crosslinked uricase is co-conjugated with both fatty acid-PEG derivatives and alkoxy-PEG derivatives.

In one embodiment, the uricase is from any source of fungal, microbial, mammalian or recombinant.

The present invention may comprise crosslinked uricase as well as native uricase.

The uricase may be intramolecularly crosslinked, which can be referred to “crosslinked uricase”.

The crosslinked uricase may be produced by reacting uricase with a crosslinking agent such as, but not limited to, bis(3,5-dibromosalicyl) fumarate, bis(3,5-dibromosalicyl) succinate, bis(3,5-dibromosalicyl) adipate, disuccinimidyl succinate, disuccinimidyl glutarate, or disuccinimidyl adipate.

In the context of the present invention, the crosslinked uricase may be applied to all embodiments of uricase.

In one embodiment, the fatty acid-PEG derivatives or alkoxy-PEG derivatives may be covalently linked to uricase or crosslinked uricase. The uricase may be conjugated via a biologically stable, nontoxic, covalent linkage to alkoxy-PEG or fatty acid-PEG. Such linkages may include, but are not limited to, urethane linkages, carbamate linkages, carbonate linkages, ester linkages, carbonyl linkages, succinimidyl linkages, secondary amine linkages or amide linkages.

In one embodiment, the one side or both sides of terminal end-groups in an alkoxy-PEG derivative and a FA-PEG derivative may be modified to contain a reactive functional group, and specifically, electrophilic functional group to be readily conjugated with uricase or crosslinked uricase.

Fatty acid-PEG derivatives and optionally, alkoxy-PEG derivatives can be conjugated with uricase or crosslinked uricase by reaction of the reactive functional group of fatty acid-PEG derivatives and optionally, alkoxy-PEG derivatives with functional group of uricase or crosslinked uricase.

Fatty-acid PEG derivatives and optionally, alkoxy-PEG derivatives can be conjugated to the surface amino acid side chains such as lysine residues of uricase following known methods specifically nucleophilic substitution reaction.

The reactive functional group of the derivatives serves to link fatty acid-PEG and alkoxy-PEG to uricase, and may become non-reactive in vivo after the reaction.

The fatty acid-PEG derivatives may be prepared by nucleophilic substitution reaction. More specifically, the fatty acid-PEG may be formed by nucleophilic substitution reaction in which a fatty acid having an electrophilic functional group is attacked by a nucleophilic group of a PEG derivative (e.g. amine or thiol), resulting in the conjugation reaction between the fatty acid and the nucleophilic atoms of a PEG derivative (e.g. nitrogen of amines or sulfur of thiols), while the electrophilic functional group from a fatty acid is detached as a leaving group.

The fatty acid having an electrophilic functional group may be formed by substitution reaction of a carboxyl group of a fatty acid, resulting in substitution of a hydroxyl group of a carboxyl group with electrophilic functional group.

In one embodiment, the FA-PEG may be a fatty acid covalently linked to a molecule of PEG.

In the preferred embodiment, the alkoxy-PEG is a methoxy-PEG(mPEG).

In one embodiment, the uricase derivative may be represented by the following formula (I): [FA-PEG]ₚ-Uricase-[alkoxy-PEG]_{q} where p=1˜50, and q=0˜80, and p denotes the number of FA-PEG conjugated to uricase, and q denotes the number of alkoxy-PEG conjugated to the same uricase.

In another embodiment, the uricase derivative may be represented by the following formula (II): [FA-PEG]ₚ-xUricase-[alkoxy-PEG]_{q} where p=1˜50, and q=0˜80, and p denotes the number of FA-PEG conjugated to xUricase, and q denotes the number of alkoxy-PEG conjugated to the same xUricase.

In one embodiment, the FA-PEG derivatives may be, but not limited to, any derivatives which comprise fatty acid-PEG and can be readily reacted with uricase or crosslinked uricase to be conjugated therewith.

In one embodiment, the FA-PEG derivatives may be selected from the group consisting of FA-PEG acetaldehyde, FA-PEG propionaldehyde, FA-PEG butyraldehyde, FA-PEG maleimide, FA-PEG succinimidyl carbonate, FA-PEG succinimidyl carboxymethyl ester, FA-PEG succinimidyl glutarate, FA-PEG succinimidyl propionate, and FA-PEG succinimidyl succinate. More preferably, the FA-PEG derivatives may be FA-PEG succinimidyl carboxymethyl ester. The structural forms of the FA-PEG can be also diverse, and the examples include, but are not limited to, linear FA-PEG, branched FA-PEG, or FA-PEG with degradable linkages built within the backbone.

In one embodiment, the FA-PEG derivatives may be stearic-PEG derivatives. The stearic-PEG derivatives may be selected from the group consisting of stearic-PEG acetaldehyde, stearic-PEG propionaldehyde, stearic-PEG butyraldehyde, stearic-PEG maleimide, stearic-PEG succinimidyl carbonate, stearic-PEG succinimidyl glutarate, stearic-PEG succinimidyl propionate, stearic-PEG succinimidyl succinate, and stearic-PEG-succinimidyl carboxymethyl ester. More preferably, may be stearic-PEG-succinimidyl carboxymethyl ester.

In one embodiment, the alkoxy-PEG derivatives may be, but not limited to, any derivatives which comprise alkoxy-PEG and can be readily reacted with uricase or crosslinked uricase to be conjugated therewith.

In one embodiment, the alkoxy-PEG derivatives may be selected from the group consisting of alkoxy-PEG acetaldehyde, alkoxy-PEG propionaldehyde, alkoxy-PEG butyraldehyde, alkoxy-PEG maleimide, alkoxy-PEG succinimidyl carbonate, alkoxy-PEG succinimidyl carboxymethyl ester, alkoxy-PEG succinimidyl glutarate, alkoxy-PEG succinimidyl propionate, and alkoxy-PEG succinimidyl succinate. More preferably, the alkoxy-PEG derivatives may be alkoxy-PEG succinimidyl succinate. The structural forms of the alkoxy-PEG can be also diverse, and the examples include, but are not limited to, linear alkoxy-PEG, branched alkoxy-PEG, or alkoxy-PEG with degradable linkages built within the backbone.

In one embodiment, the alkoxy-PEG derivatives may be methoxyPEG(mPEG) derivatives. The mPEG derivatives may be selected from the group consisting of mPEG-acetaldehyde, mPEG-propionaldehyde, mPEG-butyraldehyde, mPEG-maleimide, mPEG-succinimidyl carbonate, mPEG-succinimidyl carboxymethyl ester, mPEG-succinimidyl glutarate, mPEG-succinimidyl propionate, and mPEG-succinimidyl succinate. More preferably, the alkoxy-PEG derivatives may be mPEG succinimidyl succinate.

In one embodiment, the fatty acid may be saturated fatty acid or unsaturated fatty acid.

The saturated fatty acid or the unsaturated fatty acid may have the number of carbons from 6 to 24, preferably from 10 to 24.

The saturated fatty acids can be such as, but not limited to, stearic acid, palmitic acid, myristic acid, lauric acid, capric acid or arachidic acid. The unsaturated fatty acids can be such as, but are not limited to, oleic acid, linoleic acid, myristoleic acid, palmitoleic acid, or arachidonic acid.

The fatty acid which is used to form the fatty acid-PEG derivatives may have molecular weight of about 60˜400 Da, preferably 80˜340 Da.

In one embodiment, the PEG may have molecular weight of about 1,000˜100,000 Da. Exemplary molecular weights of PEG include about 1,000 to about 100,000 Da; about 1,000 to about 80,000 Da; about 1,000 to about 70,000 Da; preferably, about 1,000 to about 50,000 Da; and more preferably, about 2,000 to about 10,000 Da.

In one specific embodiment, the uricase derivative may be represented by the following formula (III):

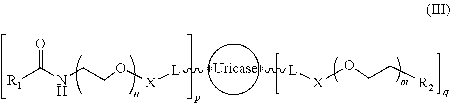

where p=1˜50, q=0˜80, n and m=each independently, 20˜2,000, R₁ is C₆₋₂₄ alkyl or C₆₋₂₄ alkenyl, R₂ is C₁₋₆ alkoxy, L is each independently NH or S, and X is each independently a divalent linker group with at least one amide, carbamate, carbonate, ester, ether, carbonyl, urethane, or succinimidyl.

In another specific embodiment, the uricase derivative is represented by the following formula (IV):

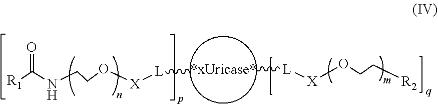

where p=1˜50, q=0˜80, n and m=each independently, 20˜2,000, R₁ is C₆₋₂₄ alkyl or C₆₋₂₄ alkenyl, R₂ is C₁₋₆ alkoxy, L is each independently NH or S, and X is each independently a divalent linker group with at least one amide, carbamate, carbonate, ester, ether, carbonyl, urethane, or succinimidyl.

In one embodiment, fatty acid-PEG and alkoxy-PEG may be covalently attached via an amino reactive moiety or sulfur reactive moiety of amino acid side chain on the uricase or crosslinked uricase. The amino reactive moiety or sulfur reactive moiety of amino acid side chain may be linked to the PEG by group X. That is, X refers to the divalent linker group which links alkoxy-PEG and uricase or crosslinked uricase, or links fatty acid-PEG and uricase or crosslinked uricase, and L refers to amino reactive moiety or sulfur reactive moiety of amino acid side chain on the uricase or crosslinked uricase.

X may be derived from the FA-PEG derivatives and alkoxy-PEG derivatives, and L may be derived from the uricase or crosslinked uricase.

In specific embodiment, X is selected from the group consisting of amide, carbamate, carbonate, ester, ether, carbonyl, urethane, succinimidyl, C₁₋₆ alkylene, C₁₋₆ alkylene-amide, C₁₋₆ alkylene-carbamate, C₁₋₆ alkylene-carbonate, C₁₋₆ alkylene-ester, C₁₋₆ alkylene-ether, C₁₋₆ alkylene-carbonyl, C₁₋₆ alkylene-urethane, C₁₋₆ alkylene-succinimidyl, amide-C₁₋₆ alkylene-carbonyl, carbamate-C₁₋₆ alkylene-carbonyl, carbonate-C₁₋₆ alkylene-carbonyl, ester-C₁₋₆ alkylene-carbonyl, ether-C₁₋₆ alkylene-carbonyl, carbonyl-C₁₋₆ alkylene-carbonyl, urethane-C₁₋₆ alkylene-carbonyl and succinimidyl —C₁₋₆ alkylene-carbonyl. More preferably, X of FA-PEG derivatives may be amide-C₁₋₆ alkylene-carbonyl and X of alkoxy-PEG derivatives may be carbonyl-C₁₋₆ alkylene-carbonyl.

In one embodiment, linkage between L and X may be formed by a nucleophilic substitution reaction in which FA-PEG derivatives or alkoxy-PEG derivatives having an electrophilic functional group is attacked by the nucleophilic groups of uricase (e.g. amines or thiols), resulting in the conjugation reaction between the FA-PEG derivatives or alkoxy-PEG derivatives and the nucleophilic atoms of uricase (e.g. nitrogen of amines or sulfur of thiols) while the functional group from FA-PEG derivatives or alkoxy-PEG derivatives is detached as a leaving group.

In one embodiment, in the case that L is S, X is succinimidyl or C₁₋₆ alkylene-succinimidyl.

In the formula (III), p and q each independently denote the number of FA-PEG conjugated to uricase and the number of alkoxy-PEG conjugated to uricase.

In the formula (IV), p and q each independently denote the number of FA-PEG conjugated to xUricase and the number of alkoxy-PEG conjugated to xUricase.

The number p is preferably 1 to 40, more preferably, 5 to 30, and most preferably, 10 to 25. The number q is preferably 0 to 60, more preferably, 5 to 50, and most preferably, 10 to 40.

In the formulas (III) and (IV), n and m are each independently denote the average number of oxyethylene units of a PEG and preferably, n and m are each independently 20 to 1,800, more preferably, 50 to 1,800, and most preferably, 100 to 1,500.

R₁ corresponds to a hydrocarbon chain which is comprised in a fatty acid. In one embodiment, R₁ is C₆₋₂₄ alkyl or C₆₋₂₄ alkenyl, preferably, R₁ is C₁₀₋₂₄ alkyl or C₁₀₋₂₄ alkenyl, more preferably, R₁ is C₁₀₋₂₂ alkyl or C₁₀₋₂₂ alkenyl, and most preferably, R₁ is C₁₀₋₂₂ alkyl. In one embodiment, in the case that R₁ is C₆₋₂₄ alkenyl, R₁ may have 1 to 5 unsaturated bonds.

In one embodiment, R₂ is C₁₋₆ alkoxy, preferably, R₂ is C₁₋₃ alkoxy, and most preferably, R₂ is methoxy.

The present invention further relates to the method for preparing the uricase derivative, comprising reacting uricase or crosslinked uricase with the FA-PEG derivatives and optionally, the alkoxy-PEG derivatives to provide uricase derivative conjugated with FA-PEG derivatives and optionally, alkoxy-PEG derivatives.

In one embodiment, reaction may be performed in a buffer solution at the temperature of 4 to 35° C. and pH of 5 to 9.

In a specific embodiment, the reaction may be performed in a buffer solution at a temperature of 4° C. to 35° C., preferably at a temperature of 10° C. to 25° C., and most preferably at a temperature of 10° C. to 20° C.

In an additional specific embodiment, the reaction may be performed in a buffer solution at a pH of 5 to 9, preferably at a pH of 6.5 to 9, preferably at a pH of 7 to 9, preferably at a pH of 7 to 8.5, and preferably at a pH of 7.5 to 8.5.

The reaction of FA-PEG derivatives and the reaction of alkoxy-PEG derivatives respectively may be performed under different conditions or may be performed in the same conditions.

After the reaction, the product may be diafiltered (e.g. using a diafiltration membrane device) and/or passed through a chromatography (e.g. ion-exchange chromatography, affinity chromatography. or size exclusion chromatography) to separate the desired product from a reactant such as unreacted uricase, unreacted FA-PEG derivatives and unreacted alkoxy-PEG derivatives and to only collect the desired product.

In one embodiment, reaction of FA-PEG derivatives and reaction of alkoxy-PEG derivatives may be progressed simultaneously or progressed in turns.

In one embodiment, uricase derivative may be prepared by reaction of uricase or crosslinked uricase first with FA-PEG derivatives, optionally, followed by reaction with alkoxy-PEG derivatives, to achieve the uricase derivative of the present invention.

More specifically, fatty acid-linked PEG (FA-PEG) may be first prepared. Next, the FA-PEG may be derivatized to possess a functional group which will allow a reaction to result in a covalent bond between the FA-PEG and the free amines of uricase or crosslinked uricase to form a FA-PEG-uricase conjugate. The reaction, in most cases, occurs via a nucleophilic substitution mechanism. As a result, the bond created between the FA-PEG derivative and the uricase or crosslinked uricase can also be diverse, and they include, but not limited to, amide, carbamate, carbonate, ester, ether or urethane linkages.

After FA-PEG-uricase conjugate is obtained, further pegylation may optionally be performed by reaction with alkoxy-PEG derivatives. The resultant product is a uricase derivative in which is co-conjugated with multiple strands of FA-PEG and alkoxy-PEG.

In another specific embodiment of the present invention, the sequence of reaction may be reversed in that the uricase derivative may be prepared by reaction of uricase or crosslinked uricase first with alkoxy-PEG derivatives, followed by reaction with FA-PEG derivatives, to achieve the identical result.

In another specific embodiment of the present invention, the uricase derivative may be prepared by reaction of uricase or crosslinked uricase with both FA-PEG derivatives and alkoxy-PEG derivatives simultaneously to achieve the identical result.

In the case of the method for preparing the uricase derivative from crosslinked uricase, firstly, crosslinked uricase may be prepared by reacting uricase with a crosslinking agent, such as, bis(3,5-dibromosalicyl) fumarate, bis(3,5-dibromosalicyl) succinate, bis(3,5-dibromosalicyl) adipate, disuccinimidyl succinate, disuccinimidyl glutarate, or disuccinimidyl adipate prior to reaction with FA-PEG derivatives and alkoxy-PEG derivatives.

The present invention provides a pharmaceutical composition comprising the uricase derivative and a pharmaceutically acceptable carrier.

Examples of a pharmaceutically acceptable carrier comprise any diluent, such as a protein, a glycoprotein, a polysaccharide, and other colloids, any salt that is pharmaceutically acceptable for delivery to a mammal, such as KCl, NaCl, NaHCO₃, NaH₂PO₄.2H₂O, MgSO₄.2H₂O, CaCl₂.2H₂O, cysteine, and dextrose.

The composition can additionally comprise pharmaceutically-acceptable fillers and other materials well-known in the art, the selection of which depends on the dosage form, the condition being treated, the particular purpose to be achieved according to the determination of the ordinarily skilled artisan in the field and the properties of such additives. For example, the composition can include a physiological buffer, a carbohydrate (e.g. glucose, mannitol, or sorbitol), alcohol or poly alcohol, a pharmaceutically acceptable salt (e.g., sodium or potassium chloride), a surfactant (e.g., polysorbate 80), an anti-oxidant, an anti-bacterial agent, an oncotic pressure agent (e.g. albumin or polyethylene glycol), or a stabilizer (e.g., ascorbic acid, glutathione, or N-acetyl cysteine).

In a preferred embodiment, the composition may include stabilizers such as, but not limited to cysteine, N-acetyl cysteine, glutathione, or ascorbate.

In a preferred embodiment, the pharmaceutical composition may be formulated in an aqueous saline solution, specifically, an aqueous isotonic saline solution, and more specifically, a physiologically acceptable electrolyte-containing aqueous isotonic saline solution.

A composition of the present invention can be administered by injecting the composition directly and/or indirectly into the circulatory system of the subject by one or more injection methods.

In one embodiment, the composition is administered simultaneously, separately, or sequentially in combination with one or more additional therapeutic agents to a subject in need thereof.

The present invention provides a method for lowering the level of uric acid comprising administering an effective uric acid-lowering amount of the uricase derivative to a subject in need thereof.

The present invention also provides a method for treating, preventing or alleviating a disease of gout or hyperuricemia, comprising administering the uricase derivative to a subject in need thereof.

The administration of the uricase derivative may be via injection by intravenous, intradermal, intramuscular, intraperitoneal or subcutaneous routes or inhalation of an aerosolized preparation.

Generally an effective administered amount of uricase derivative of the invention will depend on the relative efficacy of the uricase derivative chosen, the severity of the disorder being treated and/or prevented and the weight of the subject.

The present invention also relates to use of the uricase derivative as a medicament.

The present invention relates to use of the uricase derivative, for treating, preventing or alleviating a disease of gout or hyperuricemia.

The present invention is additionally explained below by means of examples. This explanation must by no means be interpreted as a limitation of the scope of the invention as it is defined in the claims.

### EXAMPLES

### Example 1: Preparation of a FA-PEG Derivative

In the first step of synthesis, stearic acid in organic solvent of methylene chloride was reacted with 1-ethyl-3-(3-dimethylaminopropyl)carbodiimide (EDC) and N-hydroxy succinimide (NHS), to obtain succinimidyl stearate. The reaction mechanism is illustrated below.

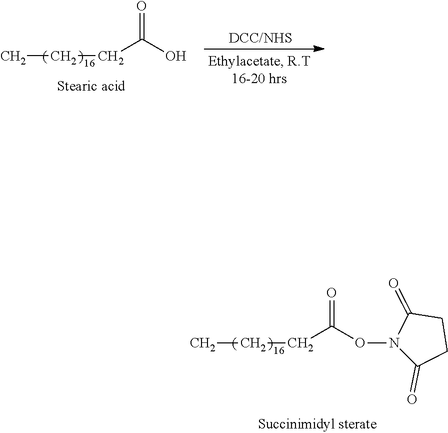

In the second step of synthesis, PEG (PEG-diol) was first reacted in organic solvent of methylene chloride (MC) with triethylamine and tosyl chloride with a subsequent addition of 28% ammonia water. In the exemplary embodiment, PEG with molecular weight of 5,000 Da is used. The intermediate PEG compound thus formed was a mixture of HO-PEG-OH (PEG-diol), amine-PEG-OH (amine PEG-alcohol), and amine-PEG-amine (PEG-diamine), shown as the compounds (1) in illustration below. This mixture was then directed to pass through an ion-exchange chromatography to separate and to only obtain amine-PEG-OH, shown as the compound (2) of illustration below. The amine-PEG-OH was then reacted in organic solvent of MC with triethylamine and di-tert-butyl dicarbonate (BOC₂O) to obtain BOC-PEG-OH, shown as the compound (3) in illustration below. The BOC-PEG-OH was then reacted with ethylisocyanoacetate (OCNCH₂CO₂-ethyl) and triethanolamine (TEA) with the addition of 1N NaOH to obtain BOC-PEG-urethane acetic acid, which was then directed to pass through an ion-exchange chromatography to obtain further purified BOC-PEG-urethane acetic acid, shown as the compound 4 in illustration below. The BOC-PEG-urethane acetic acid was reacted with trifluoroacetic acid (CF₃COOH) to remove the BOC group and finally to obtain the amine-PEG-urethane acetic acid, shown as the compound (5) in illustration below.

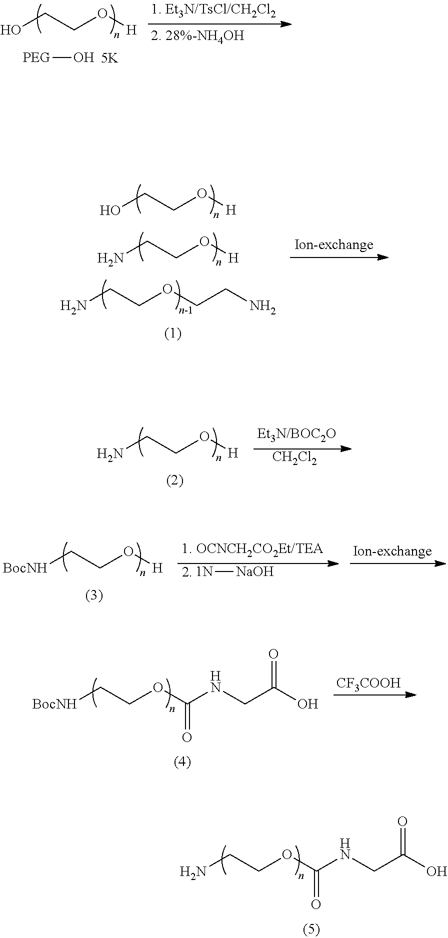

In the third step of synthesis, the succinimidyl stearate (from the first step) was reacted with amine-PEG-urethane acetic acid (from the second step) in the organic solvent of methylene chloride (MC) with the addition of base N,N-diisopropylethylamine (DIEA) under a room temperature (RT) for 16 hours to obtain the stearic-PEG-acid, as illustrated below. This compound has an acid end group, which is derivatized in the next step.

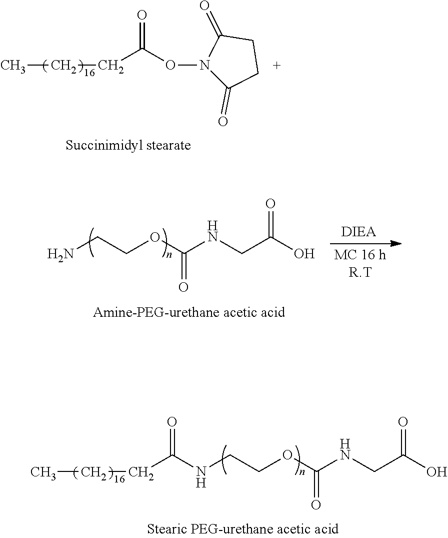

In the fourth step of synthesis, the stearic-PEG-acid obtained above was reacted in the organic solvent of MC along with N-hydroxy succinimide (NHS) and N,N-dicyclohexylcarbodiimide (DCC) under RT for 16˜20 hours to obtain stearic-PEG-urethane-succinimidyl carboxymethyl ester, as illustrated below. This stearic-PEG-urethane-succinimidyl carboxymethyl ester (Stearic-PEG-SCM) will be used to conjugate with uricase in Example 2.

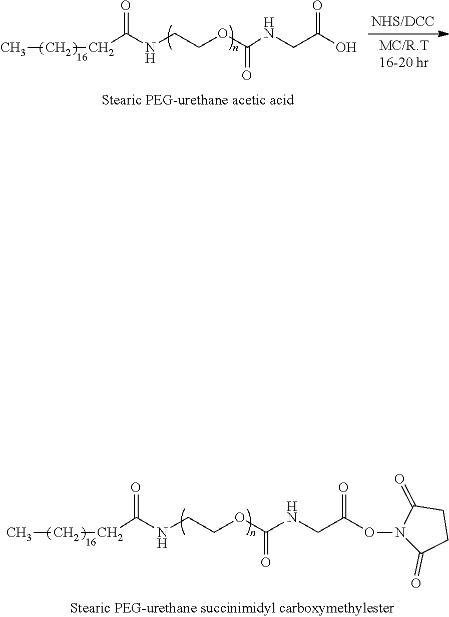

### Example 2: Preparation of [Stearic-PEG]ₚ-Uricase-[mPEG]_{q}

The uricase was reacted with stearic-PEG-urethane succinimidyl carboxymethyl ester of MW 5,000 Da (stearic-PEG-SCM) obtained from Example 1 to yield [Stearic-PEG]ₚ-uricase in the following process.

The uricase solution was prepared in an isotonic buffer solution at 15° C. at pH 8.2 with uricase concentration at 1% (w/v). Into the Uricase solution was added 20 molar equivalents of stearic-PEG-SCM and well agitated for 4 hours. As a result, [Stearic-PEG]ₚ-Uricase was obtained. The schematics of the reaction is illustrated below.

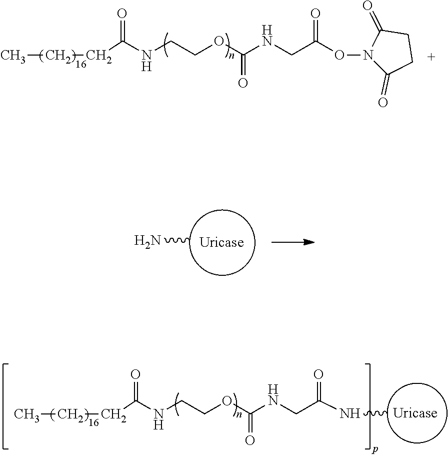

The [Stearic-PEG]ₚ-Uricase solution was directed to pass through a size exclusion chromatography to separate out and eliminate the undesired portion of the [Stearic-PEG]ₚ-Uricase, and to only collect the desired portion of the [Stearic-PEG]ₚ-Uricase, while also removing residual unreacted stearic-PEG-SCM.

The analysis to determine the number of PEGs attached to the [Stearic-PEG]ₚ-Uricase was performed by following the procedures of TNBS (2,4,6-trinitrobenzene sulfonic acid) assay method that is known in the art (Habeeb A. F.).

Based on the TNBS assay result, the number of stearic-PEG attached on [Stearic-PEG]ₚ-Uricase was 12.

Next, the [Stearic-PEG]ₚ-Uricase obtained was reacted with methoxy PEG-succinimidyl succinate of MW 5,000 Da (mPEG-SS). The [Stearic-PEG]ₚ-Uricase was prepared in isotonic buffer at pH 8.2, and at 15° C., with uricase concentration at 1% (w/v). Into the [Stearic-PEG]ₚ-Uricase solution was added an amount of mPEG-SS at 60 molar equivalents and well agitated for 4 hours. As a result, [Stearic-PEG]ₚ-Uricase-[mPEG]_{q} was obtained. The schematics of the reaction is illustrated below.

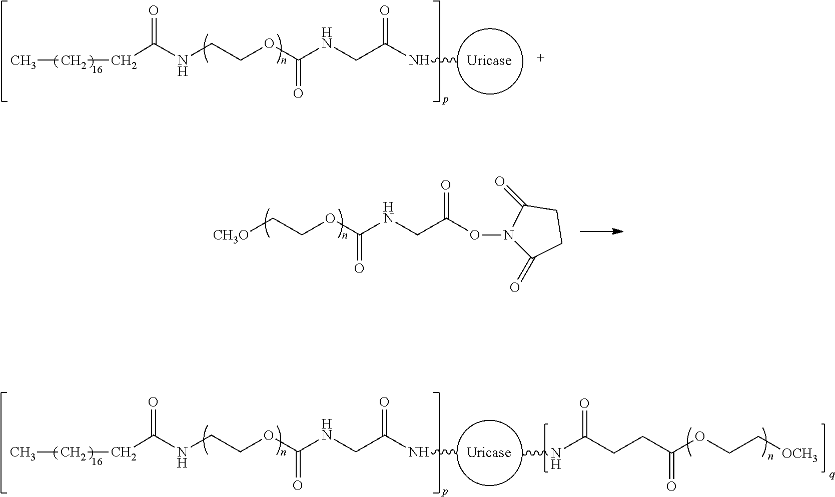

After pegylation with mPEG-SS, the resulting compound, [Stearic-PEG]ₚ-Uricase-[mPEG]_{q}, was directed through a size exclusion chromatography in order to remove unreacted mPEG-SS and to selectively obtain a portion of the final compound with a desired molecular weight. The final compound may be formulated into a physiologically acceptable electrolyte-containing aqueous isotonic saline solution, and optionally stabilizers can be added.

The analysis to determine the number of PEGs conjugated to the [Stearic-PEG]ₚ-Uricase-[mPEG]_{q} was performed by following the procedures of TNBS (2,4,6-trinitrobenzene sulfonic acid) assay method. Based on the TNBS assay result, the total number of PEG attached on [Stearic-PEG]ₚ-Uricase-[mPEG]_{q} was 43. Since this was the total number of PEG including both stearic-PEG and mPEG, and knowing that the number of stearic-PEG on the conjugate was 12, it was deduced that the number of mPEG on the conjugate was 31.

Therefore, the conjugate obtained was [Stearic-PEG]₁₂-Uricase-[mPEG]₃₁.

### Example 3: Different Methods for Preparation of [Stearic-PEG]ₚ-Uricase-[mPEG]_{q}

As illustrated in Example 2, [Stearic-PEG]ₚ-Uricase-[mPEG]_{q} was prepared by reaction of uricase first with FA-PEG derivative, followed by reaction with mPEG derivative.

However, the sequence of reaction may be reversed in that [Stearic-PEG]ₚ-Uricase-[mPEG]_{q} may be prepared by reaction of uricase first with mPEG derivative, followed by reaction with FA-PEG derivative, to achieve the identical result.

In yet another method of preparation for [Stearic-PEG]ₚ-Uricase-[mPEG]_{q}, the uricase can be reacted with both FA-PEG derivative and mPEG derivative simultaneously to achieve the identical result.

### Example 4: Preparation of [Stearic-PEG]ₚ-xUricase-[mPEG]_{q}

Intramolecularly crosslinked uricase (xUricase) was first prepared by reacting uricase with a crosslinking agent, 5 molar equivalent of bis(3,5-dibromosalicyl) fumarate, in an isotonic buffer solution at 15° C. at a pH of 8.2, with uricase concentration at 1% (w/v). Crosslinking reaction was allowed to continue for 4 hours with vigorous agitation.

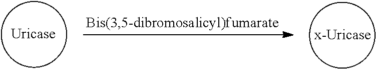

The xUricase thus obtained was then reacted with stearic-PEG-urethane succinimidyl carboxymethyl ester of MW 5,000 Da (stearic-PEG-SCM) obtained from Example 1 to yield [Stearic-PEG]ₚ-xUricase in the following process.

The xUricase solution was prepared in an isotonic buffer solution at 15° C. at a pH of 8.2, with xUricase concentration at 1% (w/v). Into the xUricase solution was added 25 molar equivalents of stearic-PEG-SCM and well agitated for 4 hours. As a result, [Stearic-PEG]ₚ-xUricase was obtained. The schematics of the reaction is illustrated below.

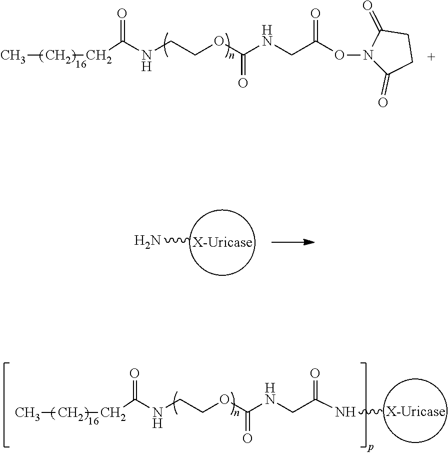

The [Stearic-PEG]ₚ-xUricase solution was directed to pass through a size exclusion chromatography to separate out and eliminate the undesired portion of the [Stearic-PEG]ₚ-xUricase, and to collect only the desired portion of the [Stearic-PEG]ₚ-xUricase, while also removing residual unreacted stearic-PEG-SCM.

The analysis to determine the number of PEGs attached to the [Stearic-PEG]ₚ-xUricase was performed by following the procedures of TNBS (2,4,6-trinitrobenzene sulfonic acid) assay method that is known in the art (Habeeb A. F.).

Based on the TNBS assay result, the number of stearic-PEG attached on [Stearic-PEG]ₚ-xUricase was 13.

Next, the [Stearic-PEG]ₚ-xUricase thus obtained was reacted with methoxy PEG-succinimidyl succinate of MW 5,000 Da (mPEG-SS). The [Stearic-PEG]ₚ-xUricase was prepared in isotonic buffer at pH 8.2, and at 15° C., with xUricase concentration at 1% (w/v). Into the [Stearic-PEG]ₚ-xUricase solution was added an amount of mPEG-SS at 70 molar equivalents and well agitated for 4 hours. As a result, [Stearic-PEG]ₚ-xUricase-[mPEG]_{q} was obtained. The schematics of the reaction is illustrated below.

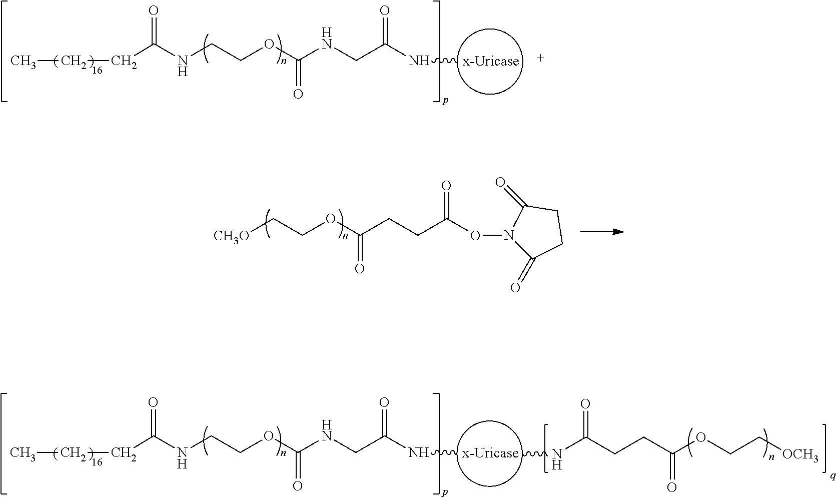

After pegylation with mPEG-SS, the resulting compound, [Stearic-PEG]ₚ-xUricase-[mPEG]_{q}, was directed through a size exclusion chromatography in order to remove unreacted mPEG-SS and to selectively obtain a portion of the final compound with a desired molecular weight. The final compound may be formulated into a physiologically acceptable electrolyte-containing aqueous isotonic saline solution, along with optionally adding stabilizers.

The analysis to determine the number of PEGs conjugated to the [Stearic-PEG]ₚ-xUricase-[mPEG]_{q} was performed by following the procedures of TNBS (2,4,6-trinitrobenzene sulfonic acid) assay method. Based on the TNBS assay result, the total number of PEG attached on [Stearic-PEG]ₚ-xUricase-[mPEG]_{q} was 46. Since this was the total number of PEG including both stearic-PEG and mPEG, and knowing that the number of stearic-PEG on the conjugate was 13, it was deduced that the number of mPEG on the conjugate was 33.

Therefore, the conjugate obtained was [Stearic-PEG]₁₃-xUricase-[mPEG]₃₃.

### Example 5: Different Methods for Preparation of [Stearic-PEG]ₚ-xUricase-[mPEG]_{q}

As illustrated in Example 4, [Stearic-PEG]ₚ-xUricase-[mPEG]_{q} was prepared by reaction of xUricase first with FA-PEG derivative, followed by reaction with mPEG derivative.

However, the sequence of reaction may be reversed in that [Stearic-PEG]ₚ-xUricase-[mPEG]_{q} may be prepared by reaction of uricase first with mPEG derivative, followed by reaction with FA-PEG derivative, to achieve the identical result.

In yet another method of preparation for [Stearic-PEG]ₚ-xUricase-[mPEG]_{q}, the uricase can be reacted with both FA-PEG derivative and mPEG derivative simultaneously to achieve the identical result.

### Example 6: Efficacy Comparison Test of [Stearic-PEG]ₚ-Uricase-[mPEG]_{q} and Native-Uricase

### Preparation Example 1. (A) Sample Preparation of [Stearic-PEG]ₚ-Uricase-[mPEG]_{q}

[Stearic-PEG]ₚ-Uricase-[mPEG]_{q} is a Uricase conjugated with both mPEG and stearic-PEG.

The Uricase (UAO-201, Toyobo Co, Ltd.) solution was prepared in an 50 mM borate buffer solution at pH 8.5 and 4° C., with concentration at 1% (w/v). Into the uricase solution was added 50 molar equivalents of mPEG-SCM (MW 10,000 Da) and well agitated for 1 hours. As a result, [mPEG]_{q}-Uricase was obtained.

After the first conjugation reaction, [mPEG]_{q}-Uricase was diafiltered against diluent of a borate buffer to remove unreacted mPEG-SCM, using a diafiltration membrane device having MW cut-off of 30,000 Da. The analysis to determine the number of PEGs attached to the [mPEG]_{q}-Uricase was performed by following the procedures of TNBS (2,4,6-trinitrobenzene sulfonic acid) assay method. Based on the TNBS assay result, the number of mPEG attached on [mPEG]_{q}-Uricase was 20.

Next, the [mPEG]₂₀-Uricase obtained was reacted with stearic-PEG-SCM (MW 5,300 Da). The [mPEG]₂₀-Uricase was prepared in 50 mM borate buffer at pH 8.5, and at 4° C., with uricase concentration at 1% (w/v). Into the [mPEG]₂₀-Uricase solution was added an amount of stearic-PEG-SCM at 50 molar equivalents and well agitated for 1 hours. As a result, [mPEG]₂₀-Uricase-[Stearic-PEG]ₚ was obtained.

After the second conjugation reaction, [mPEG]₂₀-Uricase-[Stearic-PEG]ₚ was diafiltered against diluent of an phosphate buffered saline, pH 7.4 (Gibco, 10010049) to remove unreacted stearic-PEG-SCM. The analysis to determine the number of PEGs conjugated to the [mPEG]₂₀-Uricase-[Stearic-PEG]ₚ was performed by following the procedures of TNBS (2,4,6-trinitrobenzene sulfonic acid) assay method. Based on the TNBS assay result, the total number of PEG attached on [mPEG]₂₀-Uricase-[Stearic-PEG]ₚ was 40. Since this was the total number of PEG including both mPEG and stearic-PEG, and knowing that the number of mPEG on the conjugate was 20, it was deduced that the number of stearic-PEG on the conjugate was 20. Therefore, the conjugate obtained was [mPEG]₂₀-Uricase-[stearic-PEG]₂₀.

### Preparation Example 2. (B) Sample Preparation of Native-Uricase

Native-Uricase is not Uricase conjugated with PEG derivatives.

The Uricase solution was prepared in a 50 mM borate buffer solution at pH 8.5 and at 4° C., with uricase concentration at 1% (w/v). And the uricase solution was diafiltered against diluent of a phosphate buffered saline, pH 7.4 (Gibco, 10010049), and injected for animals testing.

### Sample Analysis—Uricase Enzyme Activity Test

The enzymatic activity of Uricase preparations was performed by following the procedures of enzyme activity assay method that is known in the art (K. Itaya et al.).

The enzymatic activities of [mPEG]₂₀-Uricase-[stearic-PEG]₂₀ (A) and Native-Uricase (B) were measured. The schematics of the uricase activity assay is described as follows:

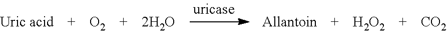

The disappearance of uric acid is measured by spectrophotometry at 290 nm. Uric acid solution was mixed with either one of the prepared Uricase samples ([mPEG]₂₀-Uricase-[stearic-PEG]₂₀ or Native-Uricase) and let it sit at 25° C. for 5 minutes, and the spectrophotometric reading was taken at 290 nm. As a result, the enzyme activity of [mPEG]₂₀-Uricase-[stearic-PEG]₂₀ (A) was 4.0 U/mg, and the enzyme activity of Native-Uricase (B) was 4.8 U/mg.

### Efficacy Evaluation of Uricase in a Hyperuricemia Rat Models

The hyperuricemia animal model in rats was prepared as follows. Male rats weighing 250˜300 g were each daily injected intraperitoneally with 500 mg/kg monosodium urate (MSU, Crysdot LLC) crystal suspension and fed a diet containing 10% yeast extract. After 2 weeks of induced hyperuricemia, the rats were randomly divided into 4 groups (n=5) and administered via tail vein 1 ml each of PBS (control group), or [mPEG]₂₀-Uricase-[stearic-PEG]₂₀ (group A), or Native-Uricase (group B), which were diluted to 4 U/ml. Daily MSU injection and high-nutrition diet were continued until the end of experiment. Blood samples were drawn from jugular vein at times points of day −1, day 0, 30 minutes, 2 hour, 8 hour, day 1, day 2, day 4, day 6, day 8, and day 10. The collected blood samples were then transferred to serum separate tubes (SST), the tubes were centrifuged at 3,500 rpm for 10 min. Isolated serum samples were analyzed for uric acid concentration using an Indiko™ Plus Clinical Chemistry analyzer (Thermo Fisher).

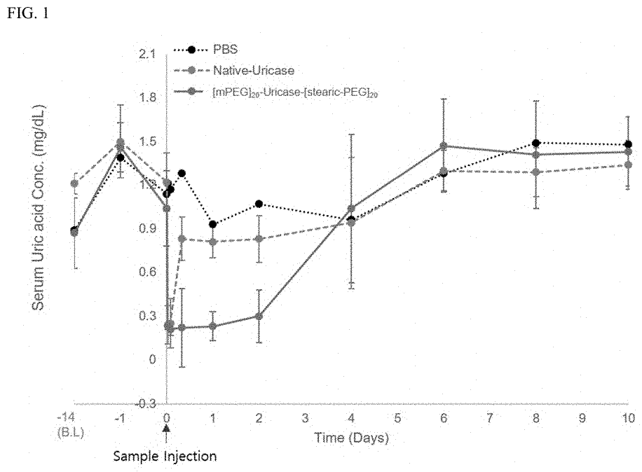

FIG. 1 shows that the in vivo uricase activity was confirmed by measuring the serum uric acid concentration after a single administration of [mPEG]₂₀-Uricase-[stearic-PEG]₂₀ (group A), and Native-Uricase (group B) to hyperuricemic rats. The mean serum uric acid concentration measured at day 0 from rats having received injection of MSU for 14 days was about 1.04 to 1.22 mg/dl. The serum uric acid level decreased to 0.3 mg/dl and maintained for 2 days in rats that received [mPEG]₂₀-Uricase-[stearic-PEG]₂₀ (group A). In contrast, the serum uric acid level decreased to 0.24 mg/dl at 2 hour, but increased to 0.83 mg/dl at 8 hour in rats that received Native-Uricase. Therefore, as shown in FIG. 1, [mPEG]₂₀-Uricase-[stearic-PEG]₂₀ (group A) demonstrated better efficacy with sustained activity than Native-Uricase (group B) in reducing the serum concentration of uric acid in the rat model in which the serum uric acid level was artificially raised by administration of MSU.

U.S. Patent Documents

| | | |
|---|---|---|
| 6,576,235 | June 2003 | Williams et al. |
| 6,783,965 | August 2004 | Sherman et al. |
| 7,723,089 | May 2010 | Williams et al. |
| 7,927,589 | April 2011 | Williams et al. |
| 7,927,852 | April 2011 | Sherman et al. |
| 8,067,553 | November 2011 | Williams et al. |
| 8,148,123 | April 2012 | Hartman et al. |
| 8,188,224 | May 2012 | Hartman et al. |
| 8,541,205 | September 2013 | Hartman et al. |
| 8,618,267 | December 2012 | Williams et al. |
| 9,017,980 | April 2015 | Hartman et al. |
| 9,193,967 | November 2015 | Zhang et al. |
| 9,670,467 | June 2017 | Hartman et al. |
| 9,885,024 | February 2018 | Williams et al. |
| 9,926,537 | March 2018 | Hartman et al. |
| 9,926,538 | March 2018 | Hartman et al. |
| 10,160,958 | December 2018 | Hartman et al. |
| 10,731,139 | August 2020 | Hartman et al. |

### OTHER PUBLICATIONS

Fields, T. R., The Challenges of Approaching and Managing Gout, Rheum Dis Clin North Am. 2019 February; 45(1):145-157.
Gout Medications, in LiverTox: Clinical and Research Information on Drug-Induced Liver Injury. Bethesda (Md.): National Institute of Diabetes and Digestive and Kidney Diseases; 2012-2017 Jul. 19.
Habeeb, A. F., Determination of Free Amino Groups in the Proteins by Trinitrobezenesulfonic Acid, Anal Biochem 14(3):328-336, 1966

What is claimed is:

1. A uricase derivative in which uricase or crosslinked uricase (xUricase) is conjugated with fatty acid-PEG (FA-PEG) derivatives and optionally, alkoxy-PEG derivatives.

2. The uricase derivative according to claim 1, wherein the alkoxy-PEG is methoxy-PEG.

3. The uricase derivative according to claim 1, wherein the FA-PEG is a fatty acid covalently linked to a molecule of PEG.

4. The uricase derivative according to claim 1, wherein the uricase derivative is represented by the following formula (I): [FA-PEG]ₚ-Uricase-[alkoxy-PEG]_{q} where p=1˜50, and q=0˜80; or the following formula (II): [FA-PEG]ₚ-xUricase-[alkoxy-PEG]_{q} where p=1˜50, and q=0˜80.

5. The uricase derivative according to claim 1, wherein the FA-PEG derivatives are selected from the group consisting of FA-PEG acetaldehyde, FA-PEG propionaldehyde, FA-PEG butyraldehyde, FA-PEG maleimide, FA-PEG succinimidyl carbonate, FA-PEG succinimidyl carboxymethyl ester, FA-PEG succinimidyl glutarate, FA-PEG succinimidyl propionate, and FA-PEG succinimidyl succinate.

6. The uricase derivative according to claim 1, wherein the alkoxy-PEG derivatives are selected from the group consisting of alkoxy-PEG acetaldehyde, alkoxy-PEG propionaldehyde, alkoxy-PEG butyraldehyde, alkoxy-PEG maleimide, alkoxy-PEG succinimidyl carbonate, alkoxy-PEG succinimidyl carboxymethyl ester, alkoxy-PEG succinimidyl glutarate, alkoxy-PEG succinimidyl propionate, and alkoxy-PEG succinimidyl succinate.

7. The uricase derivative according to claim 1, wherein the fatty acid is saturated fatty acid or unsaturated fatty acid.

8. The uricase derivative according to claim 7, wherein the saturated fatty acid or the unsaturated fatty acid has the number of carbons from 6 to 24.

9. The uricase derivative according to claim 1, wherein the PEG has molecular weight of 1,000˜100,000 Da.

10. The uricase derivative according to claim 1, wherein the uricase derivative is represented by the following the formula (III) or the formula (IV): 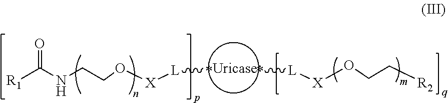
where p=1˜50, q=0˜80, n and m=20˜2,000, R₁ is C₆₋₂₄ alkyl or C₆₋₂₄ alkenyl, R₂ is C₁₋₆ alkoxy, L is each independently NH or S, and X is each independently a divalent linker group with at least one amide, carbamate, carbonate, ester, ether, carbonyl, urethane, or succinimidyl. 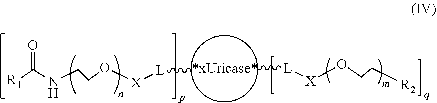
where p=1˜50, q=0˜80, n and m=20˜2,000, R₁ is C₆₋₂₄ alkyl or C₆₋₂₄ alkenyl, R₂ is C₁₋₆ alkoxy, L is each independently NH or S, and X is each independently a divalent linker group with at least one amide, carbamate, carbonate, ester, ether, carbonyl, urethane, or succinimidyl.

11. The method for preparing the uricase derivative according to claim 1, comprising reacting uricase or crosslinked uricase with FA-PEG derivatives and optionally, alkoxy-PEG derivatives to provide uricase derivative conjugated with FA-PEG derivatives and optionally, alkoxy-PEG derivatives.

12. The method according to claim 11, wherein said reaction is performed in a buffer solution at a temperature of 4 to 35° C. and a pH of 5 to 9.

13. A pharmaceutical composition comprising the uricase derivative according to claim 1 and a pharmaceutically acceptable carrier.

14. A method of treating, preventing or alleviating a disease of gout or hyperuricemia, comprising administering the uricase derivative according to claim 1 to a subject in need thereof.

15. A method according to claim 14, wherein the administration is via injection by intravenous, intradermal, intramuscular, intraperitoneal or subcutaneous routes or inhalation of an aerosolized preparation.

16. Use of the uricase derivative according to claim 1, for treating, preventing or alleviating a disease of gout or hyperuricemia.
